(19) United States
(12) Patent Application Publication
(10) Pub. No.: US 2026/0027051 A1
(43) Pub. Date: Jan. 29, 2026

(54) STABLE EMULSIONS AND METHODS OF MANUFACTURE

(71) Applicant: One Led Corp, LLC, Poulsbo, WA (US)

(72) Inventors: Leo Shlovsky, Seattle, WA (US); Yahav Blaicher, Shefer (IL); Jennifer Mohr, Poulsbo, WA (US)

(21) Appl. No.: 18/784,676

(22) Filed: Jul. 25, 2024

(51) Int. Cl. A61K 9/107; A61K 31/05; A61K 31/343; A61K 31/352; A61K 31/353; A61K 47/44; A61K 47/46; B01F 23/41

(52) U.S. Cl. CPC A61K 9/107; A61K 31/05; A61K 31/343; A61K 31/352; A61K 31/353; A61K 47/44; A61K 47/46; B01F 23/413; B01F 23/4142

(57) ABSTRACT

A stable emulsion composition (100) and method of manufacturing. The method of manufacturing can include the steps of hydrating quillaja (104), cooling the quillaja (104) to a temperature of greater than approximately 68 degrees Fahrenheit and less than approximately 72 degrees Fahrenheit, combining the quillaja (104) with an oil (106) phase mixture, adding the water-in-oil emulsion (110) to an additional volume of water (102), adding stabilizers (107) and polysaccharides that interact with emulsifiers in the water-in-oil emulsion (110) to generate a film at an oil-water interface of the water-in-oil emulsion (110), and using a high shear homogenizer to apply a shear force to the water-in-oil emulsion (110) and the additional volume of water (102), the step of using the high shear homogenizer to apply the shear force to the water-in-oil emulsion (110) and the additional volume of water (102) including a catastrophic phase inversion, the high shear homogenizer having a shear rate of at least approximately 1000 s⁻¹.

### BACKGROUND

Emulsification with complex carbohydrates leads to issues during the oil phase of the emulsification process. Often, these emulsification processes generate unstable compounds. Emulsions can face several stability issues due to their thermodynamic instability, such as coalescence, sedimentation, Ostwald ripening, and creaming. Conventional systems require additional binders and weighting agents to ensure product stability. Alternatively, to achieve a stable emulsion without these compounds, the emulsion's particle size must be reduced to a maximum of 300 nanometers, which requires the use of synthetic surfactants.

Consequently, many existing emulsification methods use synthetic surfactants to stabilize emulsions. Synthetic surfactants can sometimes have undesirable side effects or be less biocompatible compared to natural alternatives. Other problems with existing approaches include the utilization of additional weighting agents and/or carbohydrates to improve emulsion stability. Conventional technologies often rely on synthetic surfactants and simpler emulsification techniques, which may not be as effective or biocompatible as natural surfactants. Synthetic surfactants can introduce impurities and may not provide the same level of stability and quality in emulsions.

Some methods of emulsification employ standard shearing, mixing, and/or combination techniques that fail to achieve appropriate rates of oil integration, resulting in less-than-ideal consistency and efficacy of the final product containing the emulsification. Issues include broken or separated emulsions, emulsion texture, and/or flavoring issues. Furthermore, traditional emulsification methods employ simple mixing or low-shear homogenization. These methods may result in larger, less uniform particles and less stable emulsions, making it difficult to achieve a certain level of efficiency and consistency.

### SUMMARY

The present invention is directed toward a method for manufacturing a stable emulsion composition. In various embodiments, the method comprises the steps of: hydrating quillaja, cooling the quillaja to a temperature of greater than approximately 68 degrees Fahrenheit and less than approximately 72 degrees Fahrenheit, and combining the quillaja with an oil phase mixture.

In certain embodiments, the oil phase mixture includes one of Delta-7 Tetrahydrocannabinol, Delta-8 Tetrahydrocannabinol, Delta-9 Tetrahydrocannabinol, Delta-10 Tetrahydrocannabinol, Delta-11 Tetrahydrocannabinol, Cannabigerol, Cannabidiol, Cannabinol, Cannabichromene, Cannabichromenic acid, Cannabichromevarin, Cannabichromevarinic acid, Cannabicyclol, Cannabicyclolic acid, Cannabicyclolic acid, Cannabidiol monomethylether, Cannabidiolic acid, Cannabidiorcol, Cannabidivarin, Cannabidivarinic acid, Cannabielsoic acid B, Cannabielsoin, Cannabielsoin acid A, Cannabigerol monomethylether, Cannabigerolic acid, Cannabigerolic acid monomethylether, Cannabigerovarin, Cannabigerovarinic acid, Cannabinodiol, Cannabinodivarin, Cannabinol methylether, Cannabinol-C2, Cannabinol-C4, Cannabinolic acid, Cannabiorcool, Cannabivarin, 10-Ethoxy-9-hydroxy-delta-6a-tetrahydrocannabinol, 8,9-Dihydroxy-delta-6a-tetrahydrocannabinol, Cannabitriol, Cannabitriolvarin, Delta-8-tetrahydrocannabinolic acid, Delta-9-tetrahydrocannabinol-C4, acid A, Delta-9-Delta-9-tetrahydrocannabinolic tetrahydrocannabinolic acid B, Delta-9-tetrahydrocannabiorcol, Delta-9-tetrahydrocannabiorcolic acid, Delta-9-tetrahydrocannabivarin, Delta-9-tetrahydrocannabivarinic acid, 10-Oxo-delta-6a-tetrahydrocannabinol, Cannabichromanon, Cannabifuran, Cannabiglendol, Cannabiripsol, Cannbicitran, Dehydrocannabifuran, Delta-9-cis-tetrahydrocannabinol, Tryhydroxy-delta-9-tetrahydrocannabinol, and Fractionated Coconut Oil.

In some embodiments, the step of combining is completed using a high-shear homogenizer.

In various embodiments, the step of combining generates a water-in-oil emulsion including micelles composed of surfactant encapsulating water droplets within the oil phase mixture.

In certain embodiments, the method further comprises the steps of adding the water-in-oil emulsion to an additional volume of water, and applying a shear force to the water-in-oil emulsion and the additional volume of water.

In some embodiments, the additional volume of water is approximately 60% to 90% of a total volume of the stable emulsion.

In various embodiments, the step of applying the shear force to the water-in-oil emulsion and the additional volume of water includes a catastrophic phase inversion.

In certain embodiments, the step of applying the shear force includes applying a shear rate of at least approximately 1000 s⁻¹.

In some embodiments, the step of adding stabilizers, including polysaccharides that interact with emulsifiers in the water-in-oil emulsion to generate a film at an oil-water interface of the water-in-oil emulsion.

In various embodiments, the quillaja includes a quillaja saponin

The present invention is further directed toward a stable emulsion composition. In certain embodiments, the stable emulsion composition includes water, a natural surfactant including a quillaja, and an oil phase mixture that is combined with the water and the natural surfactant to generate a water-in-oil emulsion.

In some embodiments, the quillaja includes a quillaja saponin.

In various embodiments, the composition further comprises stabilizers including polysaccharides that interact with emulsifiers in the water-in-oil emulsion to generate a film at an oil-water interface of the water-in-oil emulsion.

In some embodiments, the polysaccharides include natural polysaccharides.

In various embodiments, the water is approximately 60% to 90% of a total volume of the stable emulsion.

In certain embodiments, the oil phase mixture includes one of Delta-7 Tetrahydrocannabinol, Delta-8 Tetrahydrocannabinol, Delta-9 Tetrahydrocannabinol, Delta-10 Tetrahydrocannabinol, Delta-11 Tetrahydrocannabinol, Cannabigerol, Cannabidiol, Cannabinol, Cannabichromene, Cannabichromenic acid, Cannabichromevarin, Cannabichromevarinic acid, Cannabicyclol, Cannabicyclolic acid, Cannabicyclolic acid, Cannabidiol monomethylether, Cannabidiolic acid, Cannabidiorcol, Cannabidivarin, Cannabidivarinic acid, Cannabielsoic acid B, Cannabielsoin, Cannabielsoin acid A, Cannabigerol monomethylether, Cannabigerolic acid, Cannabigerolic acid monomethylether, Cannabigerovarin, Cannabigerovarinic acid, Cannabinodiol, Cannabinodivarin, Cannabinol methylether, Cannabinol-C2, Cannabinol-C4, Cannabinolic acid, Cannabiorcool, Cannabivarin, 10-Ethoxy-9-hydroxy-delta-6a-tetrahydrocannabinol, 8,9-Dihydroxy-delta-6a-tetrahydrocannabinol, Cannabitriol, Cannabitriolvarin, Delta-8-tetrahydrocannabinolic Delta-9-acid, tetrahydrocannabinol-C4, Delta-9-tetrahydrocannabinolic acid A, Delta-9-tetrahydrocannabinolic acid B, Delta-9-tetrahydrocannabiorcol, Delta-9-tetrahydrocannabiorcolic acid, Delta-9-tetrahydrocannabivarin, Delta-9-tetrahydrocannabivarinic acid, 10-Oxo-delta-6a-tetrahydrocannabinol, Cannabichromanon, Cannabifuran, Cannabiglendol, Cannabiripsol, Cannbicitran, Dehydrocannabifuran, Delta-9-cis-tetrahydrocannabinol, Tryhydroxy-delta-9-tetrahydrocannabinol, and Fractionated Coconut Oil.

In some embodiments, the water-in-oil emulsion includes micelles composed of surfactant encapsulating water droplets within the oil phase mixture.

In various embodiments, the oil phase mixture includes a carrier oil.

In certain embodiments, the carrier oil includes a fractionated coconut oil.

The present invention is also directed toward a method for manufacturing a stable emulsion composition. In various embodiments, the method comprises the steps of hydrating quillaja, cooling the quillaja to a temperature of greater than approximately 68 degrees Fahrenheit and less than approximately 72 degrees Fahrenheit, combining the quillaja with an oil phase mixture, the step of combining includes generating a water-in-oil emulsion including micelles composed of surfactant encapsulating water droplets within the oil phase mixture, adding the water-in-oil emulsion to an additional volume of water, adding stabilizers and polysaccharides that interact with emulsifiers in the water-in-oil emulsion to generate a film at an oil-water interface of the water-in-oil emulsion, and using a high shear homogenizer to apply a shear force to the water-in-oil emulsion and the additional volume of water, the step of using the high shear homogenizer to apply the shear force to the water-in-oil emulsion and the additional volume of water including a catastrophic phase inversion, the high shear homogenizer having a shear rate of at least approximately 1000 s⁻¹.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWING

The novel features of this invention, as well as the invention itself, both as to its structure and its operation, will be best understood from the accompanying figure, taken in conjunction with the accompanying description, in which similar reference characters refer to similar parts, and in which:

FIG. 1A is a simplified illustration of one embodiment of a composition having features of the present invention, the composition being illustrated in a pre-combination state;

FIG. 1B is a simplified illustration of one embodiment of the composition illustrated in a post-combination state as a stable emulsion;

FIG. 2 is a flowchart that illustrates one embodiment of a method for manufacturing the stable emulsion composition, including processing steps and post-processing steps;

FIG. 3 is a flowchart that illustrates one embodiment of the processing steps for a method for manufacturing the stable emulsion composition; and

FIG. 4 is a flowchart that illustrates one embodiment of the post-processing steps for a method for manufacturing the stable emulsion composition.

While embodiments of the present invention are susceptible to various modifications and alternative forms, specifics thereof, have been shown by way of examples and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Embodiments of the present invention are described herein in the context of stable emulsions and methods and related methods that can be effectively utilized in food and beverage products. In particular, methods are disclosed relating to producing stable emulsions using Quillaja saponins and catastrophic phase inversion. Those of ordinary skill in the art will realize that the following detailed description of the present invention is illustrative only and is not intended to be in any way limiting. Other embodiments of the present invention will readily suggest themselves to such skilled people having the benefit of this disclosure. Reference will now be made in detail to implementations of the present invention, as illustrated in the accompanying drawings. The same or similar nomenclature and/or reference indicators will be used throughout the drawings, and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it would be appreciated that such a development effort might be complex and time-consuming. However, it would nevertheless be a routine undertaking of engineering for those of ordinary skill in the art having the benefit of this disclosure.

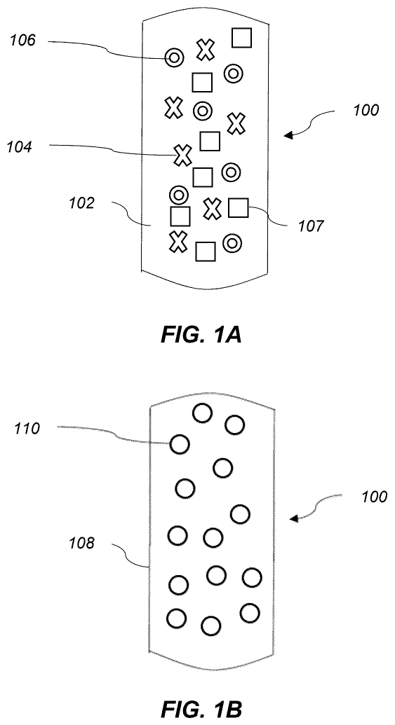

FIG. 1A is a simplified illustration of one embodiment of a composition 100, including water 102, a surfactant 104, an oil 106, and a stabilizer 107. The composition 100 illustrated in FIG. 1A displays a pre-combined state of the composition 100, including the water 102, the surfactant 104, the oil 106, and the stabilizer 107.

The composition 100 can vary depending on its design requirements. It is understood that the composition 100 can include additional elements other than those specifically shown and/or described herein. Additionally, or alternatively, the composition 100 can omit one or more of the elements that are specifically shown and/or described herein.

In various embodiments, the water 102 can be used as the base element for the composition 100. The water 102 can be mixed and/or combined with the other elements of the composition 100.

The surfactant 104 can be used to decrease the surface tension and/or interfacial tension between two or more elements within the composition 100. The surfactant 104 can include both hydrophilic and hydrophobic parts, allowing the surfactant 104 to mix the water 102 and oil 106, form foam, and aid in the removal of certain compounds.

The surfactant 104 can vary depending on its design requirements. It is understood that the surfactant 104 can include additional elements other than those specifically shown and/or described herein. Additionally, or alternatively, the surfactant 104 can omit one or more of the elements that are specifically shown and/or described herein.

The surfactant 104 can be formed from natural and/or synthetic materials. In some embodiments, the surfactant 104 is natural and includes a Quillaja, a genus of flowering plants in the family Quillajaceae. The Quillaja can include a Quillaja Saponin. The surfactant 104 can be hydrated with the water 102 and/or an additional volume of water. The surfactant 104 can be cooled and/or heated to a specific temperature prior to combination with the other elements of the composition 100. In certain embodiments, the surfactant 104 can include one of oleic acid, sunflower oil, lecithin, phosphatidylcholine, isopropyl myristate, stearic acid, medium and long chain triglycerides, Labrasol, polysorbate 20, polysorbate 80, another ethoxylated surfactant, sorbitan trioleate, and other sorbitan surfactants.

When the composition 100 is formed as an emulsion, the surfactant 104 can comprise greater than approximately 22 percent and less than approximately 32 percent relative to the total volume of the composition 100. In embodiments where the surfactant 104 includes Quillaja, this range can include greater than approximately 17 percent Saponin and less than approximately 26 percent Saponin relative to the total volume of the composition 100. In alternative embodiments, the range can extend to less than 17 percent Saponin and/or greater than 26 percent Saponin relative to the total volume of the composition.

The oil 106 can be combined with the water 102 and the surfactant 104 to generate a water-in-oil emulsion or an oil-in-water emulsion. In certain embodiments, the oil 106 can include an oil phase mixture or any varying percentage of oil and/or other elements. In many embodiments, the oil 106 and/or oil phase mixture can include active ingredients. In certain embodiments, the active ingredients included in the oil 106 and/or oil phase mixture have a purity of greater than approximately 70% for use in the composition 100 for emulsion. In alternative embodiments, the oil 106 and/or oil phase mixture can have a purity of less than 70% for use in the composition 100 for emulsion.

The oil 106 can vary depending on its design requirements. It is recognized that the oil 106 can include additional elements other than those specifically shown and/or described herein. Additionally, or alternatively, the oil 106 can omit one or more of the elements that are specifically shown and/or described herein.

For example, the oil 106 can include any suitable compound, including, but not limited to one or more of Delta-7 Tetrahydrocannabinol, Delta-8 Tetrahydrocannabinol, Delta-9 Tetrahydrocannabinol, Delta-10 Tetrahydrocannabinol, Delta-11 Tetrahydrocannabinol, Cannabigerol, Cannabidiol, Cannabinol, Cannabichromene, Cannabichromenic acid, Cannabichromevarin, Cannabichromevarinic acid, Cannabicyclol, Cannabicyclolic acid, Cannabicyclolic acid, Cannabidiol monomethylether, Cannabidiolic acid, Cannabidiorcol, Cannabidivarin, Cannabidivarinic acid, Cannabielsoic acid B, Cannabielsoin, Cannabielsoin acid A, Cannabigerol monomethylether, Cannabigerolic acid, Cannabigerolic acid monomethylether, Cannabigerovarin, Cannabigerovarinic acid, Cannabinodiol, Cannabinodivarin, Cannabinol methylether, Cannabinol-C2, Cannabinol-C4, Cannabinolic acid, Cannabiorcool, Cannabivarin, 10-Ethoxy-9-hydroxy-delta-6a-tetrahydrocannabinol, 8,9-Dihydroxy-delta-6a-tetrahydrocannabinol, Cannabitriol, Cannabitriolvarin, Delta-8-tetrahydrocannabinolic acid, Delta-9-tetrahydrocannabinol-C4, Delta-9-tetrahydrocannabinolic acid A, Delta-9-tetrahydrocannabinolic acid B, Delta-9-tetrahydrocannabiorcol, Delta-9-tetrahydrocannabiorcolic acid, Delta-9-tetrahydrocannabivarin, Delta-9-tetrahydrocannabivarinic acid, 10-Oxo-delta-6a-tetrahydrocannabinol, Cannabichromanon, Cannabifuran, Cannabiglendol, Cannabiripsol, Cannbicitran, Dehydrocannabifuran, Delta-9-cis-tetrahydrocannabinol, Tryhydroxy-delta-9-tetrahydrocannabinol, and a carrier oil such as a Fractionated Coconut Oil.

An emulsion ratio of surfactant 104 to oil 106 for the composition 100 can vary. In various embodiments, a ratio of the surfactant 104 to the oil 106 in the composition 100 herein can be between approximately 1:1 and 1:20. In some such non-exclusive embodiments, the ratio of the surfactant 104 to the oil 106 in the composition 100 can be approximately 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9, 1:10, 1:10.1, 1:10.2, 1:10.3, 1:10.4, 1:10.5, 1:10.6, 1:10.7, 1:10.8, 1:10.9, 1:11, 1:11.1, 1:11.2, 1:11.3, 1:11.4, 1:11.5, 1:11.6, 1:11.7, 1:11.8, 1:11.9, 1:12, 1:12.1, 1:12.2, 1:12.3, 1:12.4, 1:12.5, 1:12.6, 1:12.7, 1:12.8, 1:12.9, 1:13, 1:13.1, 1:13.2, 1:13.3, 1:13.4, 1:13.5, 1:13.6, 1:13.7, 1:13.8, 1:13.9, 1:14, 1:14.1, 1:14.2, 1:14.3, 1:14.4, 1:14.5, 1:14.6, 1:14.7, 1:14.8, 1:14.9, 1:15, 1:15.1, 1:15.2, 1:15.3, 1:15.4, 1:15.5, 1:15.6, 1:15.7, 1:15.8, 1:15.9, 1:16, 1:16.1, 1:16.2, 1:16.3, 1:16.4, 1:16.5, 1:16.6, 1:16.7, 1:16.8, 1:16.9, 1:17, 1:17.1, 1:17.2, 1:17.3, 1:17.4, 1:17.5, 1:17.6, 1:17.7, 1:17.8, 1:17.9, 1:18, 1:18.1, 1:18.2, 1:18.3, 1:18.4, 1:18.5, 1:18.6, 1:18.7, 1:18.8, 1:18.9, 1:19, 1:19.1, 1:19.2, 1:19.3, 1:19.4, 1:19.5, 1:19.6, 1:19.7, 1:19.8, 1:19.9, or 1:20, Alternatively, in some embodiments, the ratio of the surfactant 104 to the oil 106 in the composition 100 can be approximately can be greater than approximately 1:1 or less than approximately 1:20. In one embodiment, the ratio of the surfactant 104 to the oil 106 in the composition 100 is approximately 7:10.

The stabilizer 107 can be used to stabilize the surfactant 104 and/or resulting emulsions post-combination. In some embodiments, the stabilizer 107 interacts with emulsifiers in the emulsions of the composition 100 to generate a film at an oil-water interface of the water-in-oil emulsion or the oil-in-water emulsion. The stabilizer 107 stabilizes the texture and quality of the composition 100.

The stabilizer 107 can vary depending on its design requirements. It is understood that the stabilizer 107 can include additional elements other than those specifically shown and/or described herein. Additionally, or alternatively, the stabilizer 107 can omit one or more of the elements that are specifically shown and/or described herein.

In certain embodiments, the stabilizer 107 can include a polysaccharide. The polysaccharide can include, in non-exclusive embodiments, natural polysaccharides such as starches, vegetable gums, pectin, agar, and carrageenan.

The elements of the composition 100 can vary in percentage of weight. In one non-exclusive, representative example, the stable emulsion composition 100 that is prepared for sonication can include approximately 5% active ingredients (within the oil 106), 5% carrier oil (within the oil 106), 7% surfactants 104, and 85% water 102.

The percentage of water 102 used in the composition 100 can vary. The composition 100 can include approximately 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% water 102. Alternatively, the composition 100 can include less than approximately 1% water 102 or greater than approximately 95% water 102.

The percentage of the surfactants 104 used in the composition 100 can vary. The composition 100 can include approximately 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% surfactants 104. Alternatively, the composition 100 can include less than approximately 1% surfactants 104 or greater than approximately 95% surfactants 104.

The percentage of the oil 106 used in the composition 100 can vary. The composition 100 can include approximately 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% oil 106. Alternatively, the composition 100 can include less than approximately 1% oil 106 or greater than approximately 95% oil 106.

The percentage of the stabilizers 107 used in the composition 100 can vary. The composition 100 can include approximately 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% stabilizers 107. Alternatively, the composition 100 can include less than approximately 1% stabilizers 107 or greater than approximately 95% stabilizers 107.

FIG. 1B is a simplified illustration of one embodiment of the composition 100 in the form of a stable emulsion 108, including a water-in-oil emulsion 110. FIG. 1B illustrates the composition 100 in a post-combined state where the water 102, the surfactant 104, and the oil 106 from FIG. 1A have been combined to generate the water-in-oil emulsion 110. In various embodiments, the elements of the composition 100 can be mixed or combined together homogeneously. In certain embodiments, the mixing or combining can be completed using a high-shear homogenizer.

The stable emulsion 108 includes elements that remain uniformly dispersed. The stable emulsion 108 does not separate over time due to the presence of emulsifiers or stabilizers.

The water-in-oil emulsion 110 includes droplets of water 102 dispersed within pockets of oil 106, which surrounds and encapsulates the droplets of water 102. The compounds within the water-in-oil emulsion 110 reduce the interfacial tension between the phases, preventing coalescence and maintaining the stable emulsion's 108 integrity.

The water-in-oil emulsion 110 can include a mixed interfacial layer to induce stronger steric repulsion, thereby enhancing the emulsion's resistance to environmental stresses, including storage conditions, acidity, salinity, thermal exposure, and freeze-thaw cycles.

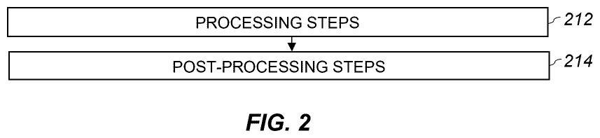

FIG. 2 is a flowchart that illustrates one embodiment of a method for manufacturing a stable emulsion composition, including processing steps and post-processing steps.

It is understood that the various steps described herein can be modified as necessary in the process of manufacturing the stable emulsion composition. Additionally, it should also be appreciated that in certain applications, the order of the steps can be modified, certain steps can be omitted, and/or additional steps can be added without limiting the intended scope and breadth of the present invention.

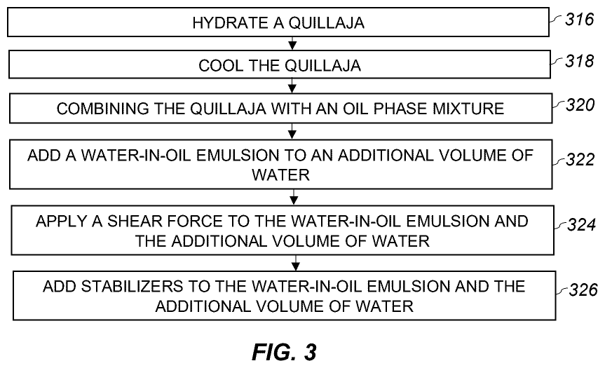

At step 212, the processing steps are performed. One non-exclusive, representative embodiment of the processing steps is illustrated and described with respect to FIG. 3.

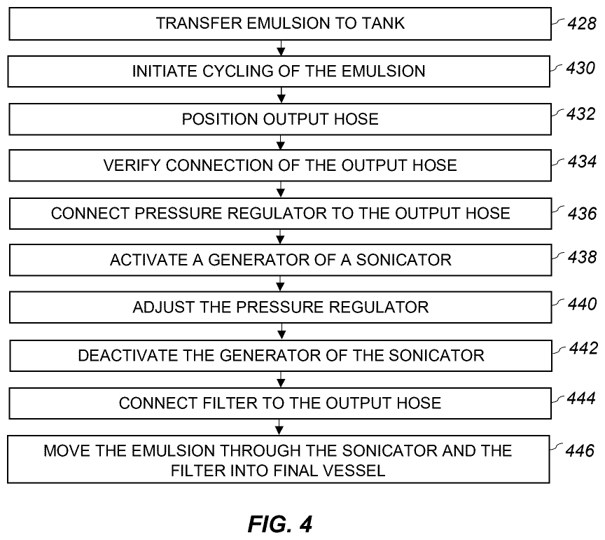

At step 214, the post-processing steps are performed. One non-exclusive, representative embodiment of the processing steps is illustrated and described with respect to FIG. 4.

FIG. 3 is a flowchart that illustrates one embodiment of a method for manufacturing a stable emulsion composition. The stable emulsion composition is suitable for use in food and/or beverage products.

It is understood that the various steps described herein can be modified as necessary in the process of manufacturing the stable emulsion composition. Additionally, it should also be appreciated that in certain applications, the order of the steps can be modified, certain steps can be omitted, and/or additional steps can be added without limiting the intended scope and breadth of the present invention.

At step 316, a Quillaja is hydrated. Water is added to the Quillaja to generate a Quillaja liquid mixture. The Quillaja can include Quillaja saponins, which are natural surfactants. In certain embodiments, the Quillaja includes the natural surfactant quillaja saponin, extracted from the Quillaja saponaria Molina tree. The Quillaja can be utilized for its efficacy in emulsion formation and stabilization. The Quillaja is capable of producing minute droplets within an emulsion at reduced surfactant-to-oil ratios through the application of high-pressure homogenization techniques. The resulting Quillaja saponin-coated droplets demonstrate resistance to coalescence under a spectrum of pH values, salt concentrations, and thermal conditions.

As used herein, a “natural surfactant” refers to a surfactant comprising plant and/or animal extracts/surfactants and does not include synthetic surfactants. In other words, a natural surfactant is a surfactant taken directly from a natural source. The source may be of either plant or animal origin, and the product should be obtained by some kind of separation procedure, such as extraction, precipitation or distillation.

When hydrated, the Quillaja saponins effectively reduce the surface tension between water and oil phases, facilitating the formation of stable emulsions. This process is advantageous due to the natural origin of Quillaja saponins, which are biocompatible and less likely to cause adverse effects when compared to synthetic surfactants.

In some embodiments, the hydration of the Quillaja is completed over the course of several minutes. The slow addition of powdered Quillaja over several minutes minimizes clumping and ensures improved dissolution of the powder into a liquid.

At step 318, the Quillaja is cooled. The Quillaja can be gradually cooled and/or heated to a specific temperature prior to combination with other elements of the composition. The cooling of the Quillaja influences its behavior, improving the emulsification process, stability of the emulsion formation, and the resulting quality of the emulsion. The step of cooling leads to a more efficient emulsification process, leading to a higher quality emulsion. The step of cooling can be completed at a natural rate, at room temperature (between greater than approximately 68 degrees Fahrenheit and less than approximately 72 degrees Fahrenheit). The step of cooling can be completed without the use of additional cooling devices, methods, and/or interventions.

The preparation of Quillaja involves carefully hydrating the Quillaja saponins and allowing the solution to cool gradually. This controlled cooling process influences the molecular arrangement of the saponins, potentially enhancing their surfactant properties. As the Quillaja cools, the Quillaja saponins undergo a transition that may improve their ability to stabilize emulsions. This could be due to the formation of more uniform micelles or enhanced interaction with lipid molecules in the oil phase. Consequently, when combined with the oil phase containing cannabinoids and other compounds, the Quillaja solution facilitates the formation of a water-in-oil (W/O) emulsion with improved initial particle size and stability.

At step 320, the Quillaja is combined with an oil phase mixture. The combination can be completed using a high-shear mixer and/or a high shear homogenizer to generate a water-in-oil emulsion by forming micelles composed of the surfactant encapsulating the water droplets within the oil phase of the emulsification process. The high shear mixing and homogenization generate fine and uniform droplets by subjecting the mixture to intense mechanical forces. These forces help the saponins encapsulate water droplets within the oil phase, forming micelles that enhance emulsion stability.

Micelle formation can originate from the Saponins present in Quillaja in addition to the other surfactants used in the oil phase component. The increased solubility leads to greater oral mucosal and gastrointestinal absorption and overall bioavailability, requiring lower concentrations of the active ingredients per effective dose.

At step 322, the water-in-oil emulsion is added to an additional volume of water. In some embodiments, the additional water used can account for greater than approximately 60% and less than approximately 90% of the total volume of the final stable emulsion composition.

At step 324, a shear force is applied to the water-in-oil emulsion and the additional volume of water. The shear force can also be applied using the high shear mixer and/or the high shear homogenizer.

An extreme increase in the additional water used during the continuous phase of the emulsification process can force a Catastrophic Phase Inversion, in which the combination of high shear forces from the homogenizer and shearing forces of inverting the equilibrium of the emulsion allow for an improved initial particle size, improved emulsion stability against phase separation, and faster, more thorough integration of the oil phase and continuous phase of the emulsification process.

The process of Catastrophic Phase Inversion (CPI) leverages a dramatic change in the phase volume ratio and the application of high shear forces to generate a stable and uniform emulsion. The process enhances particle size distribution, improves emulsion stability, ensures efficient mixing, and achieves a thermodynamically stable state. By utilizing CPI, the production of emulsions, particularly those involving complex mixtures like cannabis oil emulsions, can achieve higher quality and stability, making the final product more effective and reliable.

At step 326, stabilizers are added to the water-in-oil emulsion and the additional volume of water. The stabilizers can include plant-derived biopolymers, natural compositions, and/or polysaccharides that interact with emulsifiers to form a film at the oil-water interface, thereby augmenting the emulsion's stability. Polysaccharides, characterized by their low toxicity, high biocompatibility, and biodegradability, are utilized as stabilizers in various embodiments. These plant-derived polysaccharides are noted for their stable structures, which are attributed to robust intermolecular interactions that resist deformation under pH and temperature fluctuations.

The interaction between Quillaja saponin and various stabilizers, particularly polysaccharides, is beneficial to the performance of the emulsion, which may vary depending on the molecular attributes of the stabilizers employed. These stabilizers account for the variable stability of droplets constituted by Quillaja saponin when subjected to diverse environmental stresses, including but not limited to pH, ionic strength, temperature, and supply conditions.

FIG. 4 is a flowchart that illustrates one embodiment of the post-processing steps of a method for manufacturing a stable emulsion composition.

It is understood that the various steps described herein can be modified as necessary in the process of manufacturing the stable emulsion composition. Additionally, it should also be appreciated that in certain applications, the order of the steps can be modified, certain steps can be omitted, and/or additional steps can be added without limiting the intended scope and breadth of the present invention.

At step 428, an emulsion is transferred to a tank. In some embodiments, the tank can include a conical tank.

At step 430, cycling of the emulsion is initiated. The step of cycling can be completed using a positive displacement pump. The positive displacement pump can cycle the emulsion through connected tubing.

At step 432, an output hose is positioned. The output hose can be positioned at or slightly below an emulsion level of the emulsion inside of the tank, in order to minimize splashing and undue agitation.

At step 434, the connection of the output hose is verified. The step of verifying can include ensuring there are no kinks or air pockets in the output hose and/or any connected tubing. If obstructions to the output hose and/or connected tubing exist, the obstructions are rectified before the emulsion is moved through the output hose and/or connected tubing.

At step 436, a pressure regulator is connected to the output hose. In certain embodiments, the pressure regulator is monitored to ensure that it does not exceed certain pressure levels. In some embodiments, the pressure regulator can be set so that the pressure level does not exceed two bars or 200,000 pascals.

At step 438, a generator of a sonicator is activated. In certain embodiments, the generator of the sonicator can be set to a wattage of greater than approximately 700 watts and less than approximately 900 watts.

At step 440, the pressure regulator is adjusted. The adjustment can be made to reduce flow, increase pressure in a reaction chamber, increase wattage, and/or increase the pressure level to two bars or higher.

At step 442, the generator of the sonicator is deactivated. Even after the generator of the sonicator has been deactivated, the pump can continue cycling the emulsion until the commencement of a filtration step.

At step 444, a filter is connected to the output hose. In some embodiments, the filter can include a one-micron filter. The filter can be used to ensure the particle sizes throughout the emulsion adhere to a specific size.

An open end of the output hose can be positioned into a final vessel for holding the emulsion. During the step of connecting the filter, the pump can be deactivated.

At step 446, the emulsion is moved through the sonicator and the filter into the final vessel. The pump can be reactivated to complete the step of moving the emulsion.

The stable emulsion composition and methods of manufacture can provide a number of benefits, which can include one or more of the following:
(1) Improved Emulsification: By utilizing Quillaja tea, which leverages the natural surfactant properties of Quillaja saponins, the methods improve the emulsification of the oil phase. The gradual cooling of the Quillaja tea enhances the behavior of the saponins, leading to better emulsification compared to other methods that use complex carbohydrates.
(2) Enhanced Emulsion Stability: The inclusion of a wide range of cannabinoids and other compounds in the oil phase, combined with Quillaja tea and a carrier oil, generates a stable water-in-oil emulsion. This stability is further reinforced by the process of Catastrophic Phase Inversion, which improves the initial particle size and prevents phase separation. Catastrophic Phase Inversion (CPI) helps achieve a finer and more uniform particle size distribution. The high shear forces involved in CPI ensure that the emulsion has smaller, more evenly distributed droplets, contributing to better texture and stability of the final product.
(3) Efficient Production Process: The use of high shear mixing and homogenization techniques in the methods ensures a more efficient production process. Additionally, the use of Quillaja saponins as natural surfactants significantly reduces surface tension between oil and water phases, facilitating a more efficient and effective emulsification process. This leads to the formation of stable, uniform micelles that enhance the quality and consistency of the emulsion. The formation of micelles in the water-in-oil emulsion and the subsequent addition of water under high shear forces facilitate a thorough and rapid integration of the oil and continuous phases.
(4) Higher Quality Emulsion: The overall process, including the pre-production step of hydrating and cooling Quillaja, results in a higher-quality emulsion. This can lead to improved consistency and efficacy of the final stable emulsion composition.
(5) Fine Particle Size Distribution: Catastrophic Phase Inversion (CPI) helps achieve a finer and more uniform particle size distribution. The high shear forces involved in CPI ensure that the emulsion has smaller, more evenly distributed droplets, contributing to better texture and stability of the final product.
(6) Long-Term Stability: The combination of Quillaja tea and CPI results in an emulsion that is highly resistant to phase separation over time. This stability is critical for ensuring the shelf life and effectiveness of the emulsion, particularly in products where consistent quality is essential.
(7) Resistance to Environmental Stress: The stable emulsions produced through this method can withstand variations in temperature, pH, and other environmental factors better than those produced with conventional techniques. This robustness enhances the practical applications of the emulsion in various industries.
(8) Safety and Biocompatibility: Utilizing Quillaja saponins, which are natural and biocompatible, reduces the risk of adverse reactions compared to synthetic surfactants. This makes the emulsion safer for use in food, pharmaceuticals, and cosmetics.
(9) Environmental Benefits: The use of natural surfactants aligns with the growing demand for eco-friendly and sustainable products. It reduces the environmental impact associated with the production and disposal of synthetic chemicals.
(10) Consistency and Efficacy: The thorough mixing and fine particle size achieved through CPI lead to a more consistent product. This consistency is crucial for ensuring the efficacy of the emulsion in delivering active ingredients, such as cannabinoids, in pharmaceuticals and nutraceuticals.

It is understood that although a number of different embodiments of the stable emulsion compositions and methods have been illustrated and described herein, one or more features of any one embodiment can be combined with one or more features of one or more of the other embodiments, provided that such combination satisfies the intent of the present invention.

While a number of exemplary aspects and embodiments of the stable emulsion compositions and methods have been shown and disclosed herein above, those of skill in the art will recognize certain modifications, permutations, additions, and sub-combinations thereof. It is therefore intended that the stable emulsion compositions and methods shall be interpreted to include all such modifications, permutations, additions, and sub-combinations as are within their true spirit and scope, and no limitations are intended to the details of construction or design herein shown.

What is claimed is:

1. A method for manufacturing a stable emulsion composition, the method comprising the steps of:
hydrating quillaja;
cooling the quillaja to a temperature of greater than approximately 68 degrees Fahrenheit and less than approximately 72 degrees Fahrenheit; and
combining the quillaja with an oil phase mixture.

2. The method of claim 1 wherein the oil phase mixture includes one of Delta-7 Tetrahydrocannabinol, Delta-8 Tetrahydrocannabinol, Delta-9 Tetrahydrocannabinol, Delta-10 Tetrahydrocannabinol, Delta-11 Tetrahydrocannabinol, Cannabigerol, Cannabidiol, Cannabinol, Cannabichromene, Cannabichromenic acid, Cannabichromevarin, Cannabichromevarinic acid, Cannabicyclolic acid, Cannabicyclolic acid, Cannabidiol Cannabicyclol, monomethylether, Cannabidiolic acid, Cannabidiorcol, Cannabidivarin, Cannabidivarinic acid, Cannabielsoic acid B, Cannabielsoin, Cannabielsoin acid A, Cannabigerol monomethylether, Cannabigerolic acid, Cannabigerolic acid monomethylether, Cannabigerovarin, Cannabigerovarinic acid, Cannabinodiol, Cannabinodivarin, Cannabinol methylether, Cannabinol-C2, Cannabinol-C4, Cannabinolic acid, Cannabiorcool, Cannabivarin, 10-Ethoxy-9-hydroxy-delta-6a-tetrahydrocannabinol, 8,9-Dihydroxy-delta-6a-tetrahydrocannabinol, Cannabitriol, Cannabitriolvarin, Delta-8-tetrahydrocannabinolic acid, Delta-9-tetrahydrocannabinol-C4, Delta-9-tetrahydrocannabinolic acid A, Delta-9-tetrahydrocannabinolic acid B, Delta-9-tetrahydrocannabiorcol, Delta-9-tetrahydrocannabiorcolic acid, Delta-9-tetrahydrocannabivarin, Delta-9-tetrahydrocannabivarinic acid, 10-Oxo-delta-6a-tetrahydrocannabinol, Cannabichromanon, Cannabifuran, Cannabiglendol, Cannabiripsol, Cannbicitran, Dehydrocannabifuran, Delta-9-cis-tetrahydrocannabinol, Tryhydroxy-delta-9-tetrahydrocannabinol, and Fractionated Coconut Oil.

3. The method of claim 1 wherein the step of combining is completed using a high-shear homogenizer.

4. The method of claim 1 wherein the step of combining generates a water-in-oil emulsion including micelles composed of surfactant encapsulating water droplets within the oil phase mixture.

5. The method of claim 4 further comprising the steps of:
adding the water-in-oil emulsion to an additional volume of water; and
applying a shear force to the water-in-oil emulsion and the additional volume of water.

6. The method of claim 5 wherein the additional volume of water is approximately 60% to 90% of a total volume of the stable emulsion.

7. The method of claim 5 wherein the step of applying the shear force to the water-in-oil emulsion and the additional volume of water includes a catastrophic phase inversion.

8. The method of claim 5 wherein the step of applying the shear force includes applying a shear rate of at least approximately 1000 s⁻¹.

9. The method of claim 4 further comprising the step of adding stabilizers including polysaccharides that interact with emulsifiers in the water-in-oil emulsion to generate a film at an oil-water interface of the water-in-oil emulsion.

10. The method of claim 1 wherein the quillaja includes a quillaja saponin.

11. A stable emulsion composition comprising:
water;
a natural surfactant including a quillaja; and
an oil phase mixture that is combined with the water and the natural surfactant to generate a water-in-oil emulsion.

12. The composition of claim 11 wherein the quillaja includes a quillaja saponin.

13. The composition of claim 11 further comprising stabilizers including polysaccharides that interact with emulsifiers in the water-in-oil emulsion to generate a film at an oil-water interface of the water-in-oil emulsion.

14. The composition of claim 13 wherein the polysaccharides include natural polysaccharides.

15. The composition of claim 11 wherein the water is approximately 60% to 90% of a total volume of the stable emulsion.

16. The composition of claim 11 wherein the oil phase mixture includes one of Delta-7 Tetrahydrocannabinol, Delta-8 Tetrahydrocannabinol, Delta-9 Tetrahydrocannabinol, Delta-10 Tetrahydrocannabinol, Delta-11 Tetrahydrocannabinol, Cannabigerol, Cannabidiol, Cannabinol, Cannabichromene, Cannabichromenic acid, Cannabichromevarin, Cannabichromevarinic acid, Cannabicyclol, Cannabicyclolic acid, Cannabicyclolic acid, Cannabidiol monomethylether, Cannabidiorcol, Cannabidivarin, Cannabidiolic acid, Cannabidivarinic acid, Cannabielsoic acid B, Cannabielsoin, Cannabielsoin acid A, Cannabigerol monomethylether, Cannabigerolic acid, Cannabigerolic acid monomethylether, Cannabigerovarin, Cannabigerovarinic acid, Cannabinodiol, Cannabinodivarin, Cannabinol methylether, Cannabinol-C2, Cannabinol-C4, Cannabinolic acid, Cannabiorcool, Cannabivarin, 10-Ethoxy-9-hydroxy-delta-6a-tetrahydrocannabinol, 8,9-Dihydroxy-delta-6a-tetrahydrocannabinol, Cannabitriol, Cannabitriolvarin, Delta-8-tetrahydrocannabinolic acid, Delta-9-tetrahydrocannabinol-C4, Delta-9-tetrahydrocannabinolic acid A, Delta-9-tetrahydrocannabinolic acid B, Delta-9-tetrahydrocannabiorcol, Delta-9-tetrahydrocannabiorcolic acid, Delta-9-tetrahydrocannabivarin, Delta-9-tetrahydrocannabivarinic acid, 10-Oxo-delta-6a-tetrahydrocannabinol, Cannabichromanon, Cannabifuran, Cannabiglendol, Cannabiripsol, Cannbicitran, Dehydrocannabifuran, Delta-9-cis-tetrahydrocannabinol, Tryhydroxy-delta-9-tetrahydrocannabinol, and Fractionated Coconut Oil.

17. The composition of claim 11 wherein the water-in-oil emulsion includes micelles composed of surfactant encapsulating water droplets within the oil phase mixture.

18. The composition of claim 11 wherein the oil phase mixture includes a carrier oil.

19. The composition of claim 18 wherein the carrier oil includes a fractionated coconut oil.

20. A method for manufacturing a stable emulsion composition, the method comprising the steps of:
hydrating quillaja;
cooling the quillaja to a temperature of greater than approximately 68 degrees Fahrenheit and less than approximately 72 degrees Fahrenheit;
combining the quillaja with an oil phase mixture, the step of combining includes generating a water-in-oil emulsion including micelles composed of surfactant encapsulating water droplets within the oil phase mixture;
adding the water-in-oil emulsion to an additional volume of water;
adding stabilizers and polysaccharides that interact with emulsifiers in the water-in-oil emulsion to generate a film at an oil-water interface of the water-in-oil emulsion; and
using a high shear homogenizer to apply a shear force to the water-in-oil emulsion and the additional volume of water, the step of using the high shear homogenizer to apply the shear force to the water-in-oil emulsion and the additional volume of water including a catastrophic phase inversion, the high shear homogenizer having a shear rate of at least approximately 1000 s⁻¹.
